(19) United States
(12) Patent Application Publication
(10) Pub. No.: US 2011/0125254 A1
(43) Pub. Date: May 26, 2011

(54) DRUG RELEASING MEMBRANE FOR STENT AND DRUG RELEASING STENT FOR EXPANDING INTRALUMINAL COMPRISING THE SAME

(75) Inventors: Sung-Gwon Kang, Gyunggi-do (KR); Se-Chul Lee, Seoul (KR); Seung-Hwan Jaegal, Seoul (KR); Woo-Sung Yang, Seoul (KR); Don-Haeng Lee, Seoul (KR)

(73) Assignee: S & G BIOTECH, INC., Seongnam-si, Gyeonggi-do (KR)

(21) Appl. No.: 12/674,141

(22) PCT Filed: Oct. 30, 2007
(86) PCT No.: PCT/KR07/05389

§ 371 Date: Feb. 18, 2010

(30) Foreign Application Priority Data
Aug. 21, 2007 (KR) 10-2007-0083806

(51) Int. Cl. A61F 2/82

(52) U.S. Cl. 623/1.42

(57) ABSTRACT

The present invention relates to a drug releasing membrane for stent and a drug releasing stent for intraluminal expansion comprising the same. The drug releasing membrane according to the present invention has a two layer structure consisting of an inner layer M1 and an outer layer M2, the inner layer M is a thermosetting resin layer, and said outer layer M2 is a thermoplastic resin layer containing drug particles. In addition, the drug releasing stent for intraluminal expansion according to the present invention has a structure in which said drug releasing membrane M is inserted in a cylindrical stent body S woven with shape memory alloy wire. The present invention has excellent physical properties in spite of contact with bile during use, and is also excellent in drug therapeutic effect since drug is released only in one direction toward the skin.

### TECHNICAL FIELD

The present invention relates to a drug releasing membrane and a drug releasing stent for intraluminal expansion comprising the same, more specifically to a drug releasing membrane and a drug releasing stent for intraluminal expansion comprising the same which excels in the drug therapy effect because its physical properties are excellent in spite of contact with bile, etc. during use and drug is smoothly released only in one direction toward the skin.

Meanwhile, recently to enhance the effect of therapeutic treatment using stent, a drug releasing stent provided with a drug releasing function has been developed and used.

### BACKGROUND ART

In general, lumens in the human body can become stenosed by diseases occurring in the human body, so that the function is lowered or no functions are possible in serious cases. For example, the esophagus is stenosed due to esophageal cancer, smooth blood circulation is not possible due to arteriosclerosis, or the track for bile from liver to flow is stenosed.

In such cases, the stenosed lumen should be expanded or the expanded lumen should be prevented from becoming narrow again. As a method for expanding the stenosed passageway and maintaining it in such a case, there is a method of inserting a so-called stent into the lumen.

Normally, as a stent for intraluminal expansion, a cylindrical stent woven with shape memory alloy so as to have a plurality of space portions is generally used.

As a conventional drug releasing stent, Korean Patent Registration No. 0455343 discloses a drug releasing stent in which are formed coating layers of polyurethane and polyethylene glycol containing drug particles in a cylindrical body formed with metal wire.

As another conventional drug releasing stent, a drug releasing stent, in which a membrane of polyurethane containing drug particles in a cylindrical body formed with metal wire, is also known.

However, the conventional drug releasing stents as mentioned above have a problem that although drug is released well the physical properties such as strength are greatly lowered due to contact with bile, etc. during use, because the main components of the coating layer or membrane is polyurethane resin of a structure in which hard segments and soft segments are repeatedly arrayed.

As yet another conventional drug releasing stent, a drug releasing stent, in which a membrane of silicon resin containing drug in a cylindrical body formed with metal wire is inserted, is also known. Since such a conventional drug releasing stent is made of insoluble silicon resin, a problem of the physical properties being lowered due to contact with bile, etc. can be effectively solved. But there are problems that the structure of silicon resin is solid so drug is not released smoothly, and that when silicon resin is hardened at high temperature the drug degenerates.

### DETAILED DESCRIPTION OF THE INVENTION

### Problem to be Solved by the Invention

The present invention is to solve the above mentioned problems with an object to provide a drug releasing membrane which excels in the drug therapeutic effect because its physical properties are excellent in spite of contact with bile, etc. during the use and at the same time drug is released smoothly only in the direction of the skin.

Another object of the present invention is to provide a drug releasing stent in which a drug releasing membrane is inserted in a cylindrical stent body woven with wire.

### Technical Solution

To achieve the above objects, there is provided a drug releasing membrane for stent having a two layer structure comprising an inner layer M1 and in outer layer M2, wherein said inner layer M1 is a thermosetting resin layer and said outer layer M2 is a thermoplastic resin layer containing drug particles.

### Advantageous Effect

The present invention has excellent physical properties in spite of contact with bile, etc. during use, and also the drug therapeutic effect is excellent since drug is released smoothly only in one direction toward the skin.

### BRIEF DESCRIPTION OF THE DRAWINGS

These and other objects, features, aspects, and advantages of preferred embodiments of the present invention will be more fully described in the following detailed description, taken in conjunction with the accompanying drawings. In the drawings:

FIG. 1 is a photograph of a drug releasing stent for intraluminal expansion according to the present invention;

FIG. 2 is a sectional view schematically showing one example of the drug releasing stent for intraluminal expansion according to the present invention;

FIG. 3 is a photograph showing the state of the cross section before drug is released from the drug releasing membrane M for stent according to the present invention;

FIG. 4 is a photograph showing the surface of the outer layer M2 of the drug releasing membrane M for stent of FIG. 3 before drug is released;

FIG. 5 is a photograph showing the state of the cross section after drug is released from the drug releasing membrane M for stent according to the present invention;

FIG. 6 is a photograph showing the surface of the outer layer M2 of the drug releasing membrane M for stent of FIG. 5 after drug is released; and

FIG. 7 and FIG. 8 are schematic views showing the structure of the cylindrical stent body S composing the drug releasing stent for intraluminal expansion according to the present invention.

### DESCRIPTION OF SYMBOLS FOR MAJOR PARTS IN DRAWINGS

| | |
|---|---|
| S: cylindrical stent body | M: drug releasing membrane |

M1: inner layer of drug releasing membrane
M2: outer layer of drug releasing membrane

| | |
|---|---|
| A: inner stent | B: outer stent |
| C: fixing thread | 1: first shape memory alloy wire |
| 2: second shape memory alloy wire | |
| 1a: straight-line portion | 1b: peak portion |
| 1c: valley portion | 1d: space portion |

### BEST MODE FOR CARRYING OUT THE INVENTION

Below will be described in detail a preferred embodiment of the present invention with reference to the accompanying drawings.

First, the drug releasing membrane for stent according to the present invention has a two layer structure consisted of an inner layer M1 and an outer layer M2, characterized in that the inner layer M1 is a thermosetting resin layer and the outer layer M2 is a thermoplastic resin layer containing drug particles.

Specifically, the drug releasing membrane M for stent according to the present invention is a two layer structure made of the inner layer M1, which is a thermosetting resin layer, and the outer layer M2, which is a thermoplastic resin layer containing drug particles.

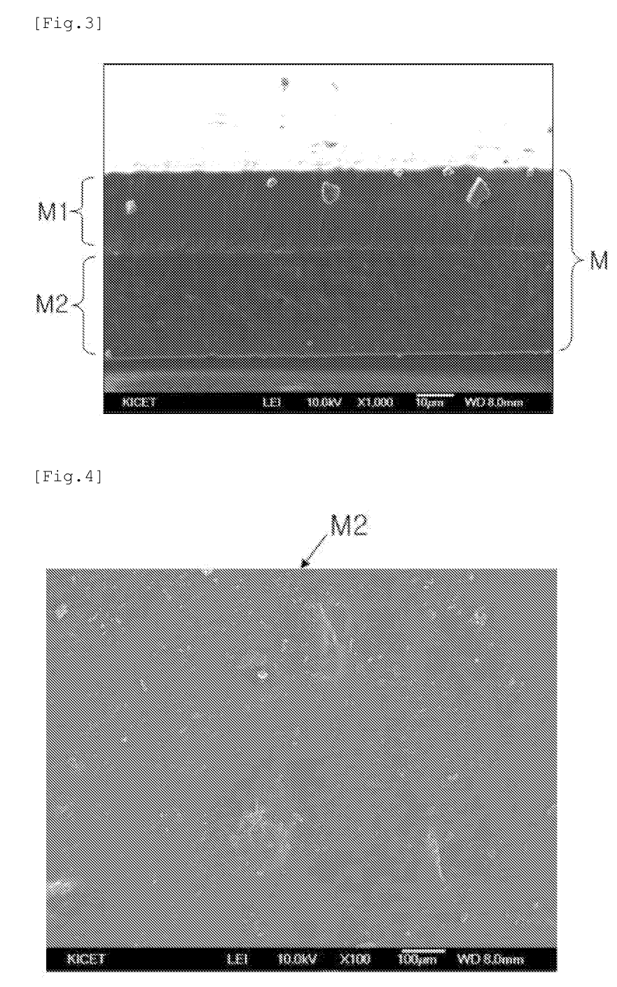

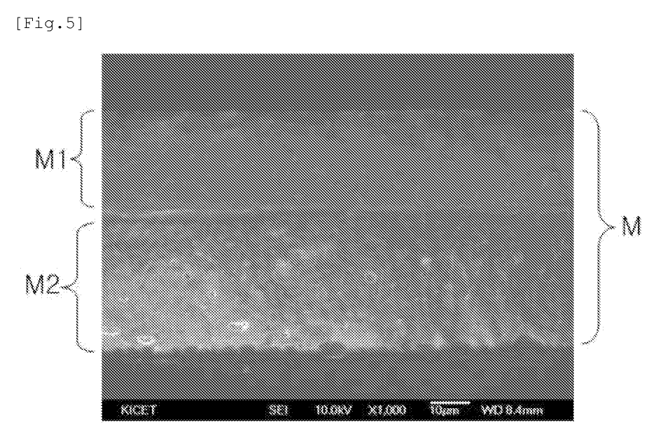

FIG. 2 is a sectional view schematically showing a drug releasing stent according to the present invention, and FIG. 3 and FIG. 5 are photographs showing respectively the cross-sectional conditions before and after the drug releasing membrane M releases drug.

Silicon resin, etc. can be used for the thermosetting resin of the inner layer M1 and polyurethane resin, etc. can be used for the thermoplastic resin of the outer layer M2.

If surgery is undergone in the human body, the inner layer M1 is adhered to the cylindrical stent body S for primary contact with secretions such as bile secreted from lumen and the outer layer M2 comes into direct contact with the skin surface.

As mentioned above, the present invention has solved the problem of the physical properties being lowered during use by composing the inner layer M1 that is in direct contact with bile, etc. during the use, with silicon resin, which is an insoluble resin.

Also, the outer layer M2 that is in direct contact with the skin during the use of the present invention is composed of polyurethane resin containing the drugs, so that the therapeutic effect is enhanced by making the drug release smoothly only in one direction toward the skin.

The outer layer M2 may further comprise polyethylene glycol and/or antimicrobial agent.

The drug particles are any one of anticancer drug particles, biological immune enhancer, and mixture thereof, and the kinds of drug particles are not, specially limited thereto in the present invention.

If relatively smallest drug particles are contained in the inside and relatively largest drug particles are contained in the outside so that the particle size of contained drug gradually increases as it goes from the inside to the outside of the outer layer M2, it is all the more effective to improve the drug releasing effect during use.

Meanwhile, the drug releasing stent for intraluminal expansion according to the present invention comprises a cylindrical stent body S woven with shape memory alloy wire and a drug releasing membrane M inserted in the cylindrical stent body S. In such a drug releasing stent for intraluminal expansion, the drug releasing membrane M is a two layer structure consisted of the inner layer M1 and outer layer M2, and characterized in that the inner layer M1 is a thermosetting resin layer, and the outer layer M2 is a thermoplastic resin layer containing drug particles.

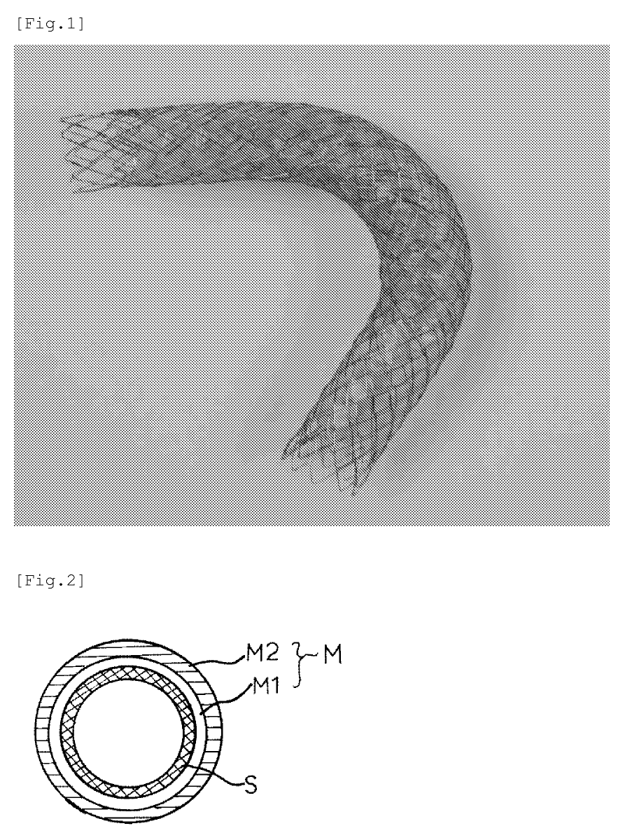

In other words, the drug releasing stent of the present invention, as shown in FIG. 1, is characterized in that a drug releasing membrane M, which is the aforementioned two layer structure of the present invention, is inserted in the cylindrical body S.

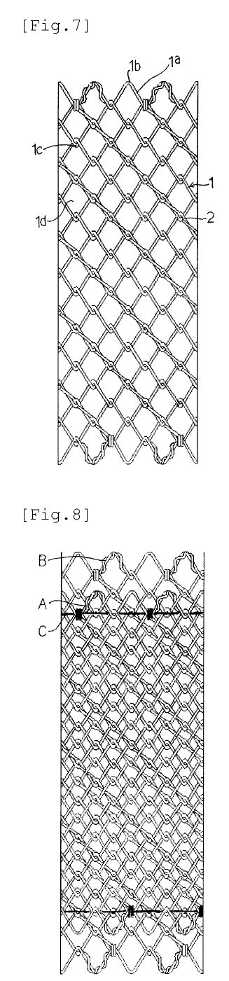

FIG. 1 is a photograph of a drug releasing sent for intraluminal expansion according to the present invention. As the above mentioned cylindrical stent body S, the stent bodies illustrated in FIG. 7 and FIG. 8 can be used.

FIG. 7 and FIG. 8 are schematic views showing the structure of such cylindrical stent bodies S.

As an example, in the cylindrical stent body S, as shown in FIG. 7, a first shape memory alloy wire 1 makes a plurality of turns in zigzags so as to make a plurality of straight-line portions 1a and the peak portions 1b and valley portions 1c that connect said straight-line portions 1a by a plurality of bending points, and the valley portion of any one turn and the peak portion corresponding to the turn adjacent to this one turn are entwined and connected to each other to form a plurality of space portions 1d. In the diagonal direction, a second shape memory alloy wire 2 has a structure entwined with the first shape memory alloy wire 1 at a given interval.

In the present invention, the structure of the cylindrical stent body S is not specially limited.

But in order to effectively cut off cancer cells, etc. penetrating into the lumen by decreasing the unit sizes of the space portions in the cylindrical stent body S, the cylindrical stent body S also is composed of the cylindrical inner stent A and outer stent B that are woven with shape memory alloy wires 1 and 2, and fixing threads C for fixing these stents as one body, so as to have a plurality of space portions 1d as shown in FIG. 8. And the outer stent B is inserted over the inner stent A in such a way that the space portions of the inner stent A and the space portions of the outer stent B alternate with each other, so the outer surfaces of the inner stent and the inner surfaces of the outer stent are in close contact with each other. At this time, both ends of the outer stent and the inner stent are fixed as one body by fixing threads C. Therefore, it is preferable to use a stent body of such a structure.

Also, it is preferable that the aforementioned drug releasing membrane M is installed between inner stent A and outer stent B.

Below will be described in detail an inserting device of artificial blood stent according to a preferred example of the present invention with reference to the accompanying drawings.

But the present invention is not limited to the example described below.

### Example 1

After dipping a cylindrical bar in silicon resin solution, hardening and drying processes were performed at 180° C. to form a resin coated layer M1 of thickness of 35 μm on the surface of the cylindrical bar.

Next, the cylindrical bar with silicon resin coated on the surface like above is dipped in polyurethane resin solution containing 14 mg of OK 432 5 Bial (drug) and 5 mg of gold (Ag) particles and then dried to form polyurethane resin coated layer M2 of thickness of 50 μm also containing drug over the silicon resin coated layer formed on the surface of said cylindrical bar. Next, the cylindrical bar only is separated to prepare a drug releasing membrane M for stent of a two layer structure in which the polyurethane resin layer M2 containing the drug and gold particles (antimicrobial agent) is formed over the silicon resin layer M1 as one body. Next, the drug releasing membrane M for stent is inserted between the inner stent A and the outer stent B of a cylindrical stent body S having a structure as shown in FIG. 8 to manufacture a drug releasing stent for intraluminal expansion.

The photograph of the cross section of the drug releasing membrane M for stent before drug is released is as shown in FIG. 3, and the photograph of the surface of the polyurethane resin layer M2 before drug is released is as shown in FIG. 4.

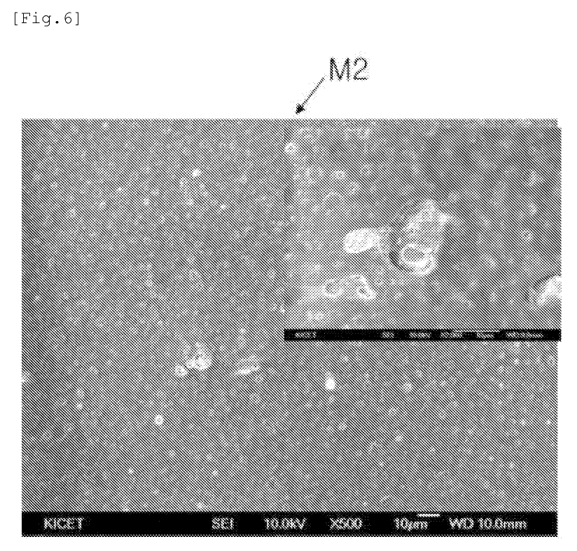

The photograph of the cross section of the drug releasing membrane M for stent that has released drug for 3 days is shown in FIG. 5, and the photograph of the surface of the polyurethane resin layer M after drug is released is shown in FIG. 6.

### INDUSTRIAL APPLICABILITY

The present invention is used to expand the stenosed lumen or prevent the expanded lumen from becoming narrow again, in case the esophagus is stenosed due to esophageal cancer, blood does not circulate smoothly due to arteriosclerosis, or the track for bile coming out from the liver to flow is stenosed.

Although the present invention has been described in connection with the exemplary embodiments illustrated in the drawings, it is only illustrative. It will be understood by those skilled in the art that various modifications and equivalents can be made to the present invention. Therefore, the true technical scope of the present invention should be defined by the appended claims.

What is claimed is:

1. A drug releasing membrane for stent having a two layer structure comprising an inner layer M1 and in outer layer M2, wherein said inner layer M1 is a thermosetting resin layer and said outer layer M2 is a thermoplastic resin layer containing drug particles.

2. The membrane of claim 1, wherein the drug particles are any one selected from the group consisting of anticancer particles, biological immune enhancer particles, and a mixture thereof.

3. The membrane of claim 1, wherein said outer layer M2 further contains antimicrobial agent particles.

4. The membrane of claim 1, wherein the size of contained drug particle gradually increases as it goes from the inside to the outside of said outer layer M2.

5. The membrane of claim 1, wherein said thermosetting resin is silicon resin.

6. The membrane of claim 1, wherein said thermoplastic resin is polyurethane resin.

7. A drug releasing stent for intraluminal expansion having a cylindrical stent body S woven with shape memory alloy wire and a drug releasing membrane M that is inserted in said cylindrical stent body S, wherein said drug releasing membrane M has a two layer structure comprising an inner layer M1 and an outer layer M2, and said inner layer M1 is a thermosetting resin layer, and said outer layer M2 is a thermoplastic resin layer containing drug particles.

8. The stent of claim 7, wherein said cylindrical stent body S is composed of cylindrical inner stent A and outer stent B woven with shape memory alloy wires 1 and 2, and fixing threads C for fixing these stents as one body, so as to have a plurality of space portions 1d, and said outer stent B is inserted over said inner stent A so that the outer surface of said inner stent and the inner surface of the outer stent are in close contact with each other, in such a way that the space portions of said inner stent A and the space portions of said outer stent alternate with each other, and both ends of said outer stent and inner stent are fixed as one body by fixing threads C.

9. The stent of claim 8, wherein a drug releasing membrane M is inserted between said inner stent A and said outer stent B.

10. The membrane of claim 7, wherein the drug panicles are any one selected from the group consisting of anticancer particles, biological immune enhancer particles, and a mixture thereof.

11. The membrane of claim 7, wherein said outer layer M2 further contains antimicrobial agent particles.

12. The membrane of claim 7, wherein the size of contained drug particle gradually increases as it goes from the inside to the outside of said outer layer M2.
